(19) United States
(12) Patent Application Publication
(10) Pub. No.: US 2024/0197935 A1
(43) Pub. Date: Jun. 20, 2024

(54) Apparatus and method for treating the inner walls of containers

(71) Applicant: KRONES AG, Neutraublig (DE)

(72) Inventor: Matthias WAHL, Langquaid (DE)

(21) Appl. No.: 18/288,020

(22) PCT Filed: Mar. 23, 2022
(86) PCT No.: PCT/EP2022/057700

§ 371 Date: Oct. 23, 2023

(30) Foreign Application Priority Data
Apr. 22, 2021 (DE) 10 2021 110 223.5

(51) Int. Cl. A61L 2/08; B65B 57/04

(52) U.S. Cl. CPC A61L 2/087; B65B 57/04; A61L 2202/14; A61L 2202/23

(57) ABSTRACT

Apparatus for treating the inner wall of containers, having a transport device which transports the containers along a predetermined transport path, wherein the containers have a mouth opening, wherein the apparatus has at least one treatment device which can be introduced into the containers through the mouth opening, wherein the apparatus has a monitoring device which monitors the treatment of the inner wall wherein the monitoring device is designed such that it monitors the treatment of the inner wall through the mouth opening.

### BACKGROUND OF THE INVENTION

The present invention relates to an apparatus and a method for treating the inner surfaces of containers. Various methods and apparatuses are known from the prior art which treat the inside walls of containers for different purposes. For example, it is known from the prior art to coat the inside walls with a plasma to avoid in this way the diffusion of substances from a beverage or also into a beverage.

In addition, apparatuses and methods are known from the prior art in which the inside walls of containers are sterilized—in particular, before the containers are filled with a beverage.

In both cases, apparatuses and methods are known from the prior art in which elements are introduced into the interior of the containers, such as lance-like sterilization bodies or also electrodes, in the case of plasma coating.

In the case of container sterilization, for example, with electron radiation or also with other radiation, monitoring of the sterilization treatment is necessary in order to be able to ensure satisfactory germ killing during operation. For example, a failure of an emitter finger or an electrode can be detected in time, wherein said emitter finger or electrode directs an electron beam or the like through the mouth into the interior of the container.

Different approaches are known from the prior art for detecting such failures. For example, DE 10 2009 018 210 B4 discloses a method for monitoring the intensity of an electron beam generating a plasma during its propagation. In this case, a detector monitors the actual working process through the wall of a transparent or translucent packaging material.

This approach has proven to be satisfactory, but in some cases has the disadvantage that falsification of this observation by the wall that is looked through can result. This approach also meets its limits in containers made of less transparent materials.

A method for examining and checking coated containers is also known from DE 10 2012 200 976 A1, wherein unwanted foreign matter emerging from the container material, e.g., antimony, is detected by a measuring device. In principle, methods for plasma sterilization are known, for example, from DE 199 09826 A1 or DE 102 366 83 A1.

Methods for electron beam sterilization are known, for example, from DE 10 2008 045 187 A1. Further internal coating methods by means of plasma are known, for example, from DE 10 2010 000 940 A1 or DE 10 2012 201 956 A1.

Based upon the cited prior art, the object is therefore to propose an apparatus and a method which enable monitoring of said treatment process without any interferences occurring—for example, due to a container wall. Furthermore, a method is to be provided which, optionally, also enables checking containers which are not or are less transparent.

### SUMMARY OF THE INVENTION

An apparatus according to the invention for treating the inner walls of containers has a transport device which transports the containers along a predetermined transport path, wherein the containers have an mouth opening and wherein the apparatus has at least one treatment device that can be introduced into the containers through said mouth opening, wherein the apparatus further has a monitoring device which monitors the treatment of the inner wall.

According to the invention, the monitoring device is designed such that it monitors and/or observes the treatment (or the treatment process) of the inner wall through the mouth opening. Preferably, said monitoring takes place at least at times during the treatment process and/or at least at times during a (treatment) emission of the treatment device.

In contrast to the prior art, it is therefore proposed that the observation of the treatment process not take place through a container wall, but through the mouth opening itself. In this way, it can be ensured that there are no erroneous results due to artifacts of the container wall. Monitoring through the mouth opening is understood in particular to mean that the event to be monitored takes place in the interior of the container, but is observed through the mouth.

It is possible, for example, for a detector device or a sensor device to be arranged in such a way that, for example, plasma formation in the interior of a container can be observed and/or monitored through the mouth. It is conceivable that said plasma serve for the sterilization and/or the inner coating of a container (such as, for example, a plastic bottle or a preform).

In a further preferred embodiment, the monitoring device is suitable and intended—in particular, in response to a signal of the sensor device—for outputting information and/or a signal characteristic of a correct or of a faulty treatment process. In a preferred embodiment, the apparatus has a rejection device which, in response to a signal of said monitoring device, causes a specific container to be rejected from a container flow, so that said container is no longer treated. In addition, however, the monitoring device can also output a warning signal.

The apparatus can also have a control device which causes no additional containers to be fed to said treatment device when a faulty treatment through a specific treatment device is detected.

In a preferred embodiment, the treatment device is rod-like and/or tubular. This means that the treatment device extends, in particular, in a preferred direction, and, in particular, in a longitudinal direction of the container to be treated. The treatment device may have one (or more) rod-like bodies, e.g., in the form of an emitter finger for applying electrons to the inner walls. In addition, however, the treatment device may also have several rod-like elements, e.g., two electrodes, which are likewise preferably introduced into the interior of the container to be treated.

In a further advantageous embodiment, the apparatus has a drive device which moves the containers with respect to the treatment device. It is thus possible for the rod-like container device to be arranged stationary in the longitudinal direction of the containers and for the containers themselves to be raised, for example, so that the treatment device can penetrate into the containers.

The transport device preferably transports the containers to be treated with the mouth thereof in the upward position. The mouth of the containers preferably has a smaller cross-section than a main body and/or a bottom of the containers.

Particularly preferably, said drive device moves the containers in their longitudinal direction so as to insert the treatment devices into the containers.

The containers are preferably plastic bottles or so-called plastic preforms. Additionally, the containers could, however, also be glass containers. The apparatus preferably has a gripping device which is suitable and intended for gripping the plastic containers below the carrier ring thereof and/or in the mouth region thereof.

In principle, it would be possible for the containers to be treated while the containers are standing still, i.e., the apparatus is operated in a cycle mode. However, it would also be possible for the treatment to take place while the containers are moving, i.e., during the transport movement of the containers along said transport path.

In a further advantageous embodiment, the transport device has a rotatable carrier on which a plurality of the treatment devices described herein are arranged.

Particularly preferably, an observation path of the observation or monitoring device runs at least in sections in the longitudinal direction of the containers and/or a longitudinal direction of the treatment device. It is possible for said observation path to run parallel to the treatment device.

In a further preferred embodiment, the monitoring device has a sensor device which is suitable and intended for detecting electromagnetic radiation emerging from the interior of the container through the mouth opening. Said sensor device can be an image recording device—for example, a camera which looks through the mouth of the containers into the interior of the containers.

The sensor device preferably has an image recording device which records at least one image from the interior of the container through its mouth opening. It is possible for said image recording device to always be arranged outside the container independently of a position of the container relative to the treatment device and, in particular, above the mouth of the container. The image recording device is preferably suitable and intended for recording an image that is characteristic of a proper treatment process on the container and/or an image of radiation produced during a treatment process in the interior of the container.

In a further advantageous embodiment, the sensor device is suitable and intended for detecting the radiation when the treatment device is introduced into the container. This means that the sensor device preferably looks into the container through a gap which is created between the treatment device and the mouth opening of the containers.

In a preferred embodiment, the sensor device is arranged axially parallel to the treatment device—for example, a radiation emitter and/or an electrode. In this case, a position above an exit window of a radiation emitter is particularly preferably advantageous.

In addition, it would also be conceivable that, for example, the treatment device has a changed—in particular, thickened—upper part, and that, in this region, a detection device or sensor device arranged at least in sections and preferably completely annularly around said region is provided.

Furthermore, it would also be possible for the treatment device to be introduced into the container slightly eccentrically with respect to a geometric longitudinal direction of the container (or an axis of symmetry of the container), so that an enlarged gap results through which the observation of the interior of the container can take place.

In a preferred embodiment, the treatment device has an emissions device which is suitable and intended for applying electron radiation or plasma to the inner wall of the containers. Particularly preferably, the treatment device is a treatment device which carries out a coating process on the container and/or a treatment device which carries out a sterilization process on the container.

In the case of a sterilization device, the treatment device can, in particular, be an emitter device and, in particular, an electron emitter device which applies electron radiation and/or electrons to the inner wall of the containers.

In this case, the treatment device preferably has an acceleration device which accelerates charge carriers and, in particular, electrons in the direction of an exit window. Said exit window is particularly preferably formed from titanium.

In this embodiment, the treatment device is preferably designed as an emitter finger.

Particularly in the case where the treatment device is a coating device, the treatment device has at least one and preferably two electrodes which can be introduced through the mouth of the container into the interior thereof. In addition, a plasma output device, such as a feed tube, is preferably introduced into the container together with the electrodes.

The above-mentioned radiation is, as mentioned above, preferably, in particular, electron radiation, but it would also be possible for other types of radiation to be used for sterilization, such as UV radiation, X-ray radiation, or similar radiation.

The sensor device is preferably suitable and intended for detecting UV radiation and/or radiation in the visible spectral range. It is possible, for example, for the sensor device (during the treatment process of the container) to detect an electromagnetic radiation which occurs during the propagation of an electron beam inside the container. The sensor device may preferably have a semiconductor sensor.

Furthermore, the sensor device may have an element which is selected from a group of elements containing photodiodes, CCD chips, light-sensitive CMOS sensors, and phototransistors. The sensor device preferably allows spatially resolved detection of the radiation impinging thereon.

In a further preferred embodiment, the apparatus has a vacuum chamber within which the treatment device is arranged.

In a further preferred embodiment, the treatment device has at least one cooling device which is suitable and intended for cooling components of the treatment device. A cooling device, for example, may be provided which cools an exit window for electrons. In addition, a cooling device which cools the electrodes may also be provided. It is possible for cooling to take place by applying a flowable medium to the respective elements—for example, sterile air. However, it would also be possible for the cooling device to be a liquid cooling device which, for example, applies a cooling medium to the electrodes. It would be possible for the electrodes to have channels through which a cooling medium can flow.

In a further advantageous embodiment, an image recording device and/or a radiation deflecting device is arranged on the treatment device. For example, it would be possible for an image recording device such as a camera to be arranged on an upper region of the treatment device, which itself is not introduced into the container. In addition, however, a radiation deflecting device such as a mirror, cone, or a prism could also be arranged, which deflects radiation onto an image recording device.

Therefore, the image recording device and/or the radiation deflecting device is preferably fixedly arranged in a vertical direction or in a longitudinal direction of the containers to be treated. Preferably, the image recording device and/or the radiation deflecting device are fixedly arranged relative to the treatment device. It is possible for the image recording device and/or the radiation deflecting device to be fixedly arranged on a predetermined section of the treatment device and, in particular, on a section which cannot be introduced or is not introduced into the container.

Particularly preferably, the observation device is aligned parallel to the treatment device, i.e., radiation impinges on the image recording device—in particular, in a direction parallel to the treatment device and, in particular, also in a direction parallel to the longitudinal direction of the containers to be treated.

In a further advantageous embodiment, the radiation deflecting device could be a mirror, cone, or a prism arranged at a 45° angle to a longitudinal axis, for example. This could preferably be used with a through hole for the electrode and/or the emitter or the treatment device, so that the detector or the image recording device can look onto the deflecting mirror substantially transversely to the longitudinal axis, and thus into the interior of the container onto a plasma.

In addition, such a radiation deflecting device or an image recording device can also be attached directly to an emitter or an electrode.

For example, as mentioned above, a camera which can detect at least electromagnetic radiation could be used as the detector or sensor device.

In a further particularly preferred embodiment, the treatment device has elements which are selected from a group of elements containing electron accelerators, electrodes, exit windows for electrodes, plasma output tubes, and the like.

In a further advantageous embodiment the monitoring device could also have a light guiding device. In this case, an end portion of said light guiding device can be arranged, for example, on the treatment device and, in particular, also a lower end, i.e., an end of the treatment device protruding into the container. Said light guiding device preferably extends through the mouth of the container in a state in which the treatment device is introduced into the container. Furthermore, it is possible for the light guiding device to be arranged at least in sections on the treatment device and/or on elements of the treatment device.

In a further advantageous embodiment, the light guiding device is designed to be electrically and/or thermally non-conductive.

In a further preferred embodiment, the light guiding device is arranged on the respective treatment stations and/or treatment devices in a stationary manner. Preferably, a light guiding device is associated with each treatment station and/or each treatment device.

In a further preferred embodiment, an optical coupling device is provided, which transmits a signal emerging from the light guiding device or devices and particularly preferably transmits it to a stationary part of the apparatus. Said coupling device is preferably designed such that an input-coupling of the signal originating from the light guiding device is transmittable to the stationary part of the apparatus in a contactless manner.

In a preferred embodiment, the coupling device can be designed to be segment-like. In said embodiment, the signal of the light guiding device preferably occurs only in one segment (for example, a movement of the treatment device), in which a treatment takes place.

In a further preferred embodiment, the transmitter can be designed as a fiber-optic rotary transmitter.

In addition, an annular detection device could also be provided, which particularly preferably extends around an opening of the container or in this region. If, as mentioned above, the treatment of the container takes place in a closed space, e.g., of a vacuum chamber, it is also possible for a detector or an image recording device to also be arranged outside said chamber. In this case, it would be possible for a sealed sight glass or lens or the like to be used to check the treatment process—for example, to check the plasma during a work cycle.

The approach proposed here of performing the observation through a mouth of the containers is unusual, because the mouths of the containers are comparatively narrow, and the observation is usually also performed with the treatment device inserted into the containers.

In a further advantageous embodiment, the monitoring device has a protection device for protecting a sensor device from emissions occurring during the treatment (for example, from electrons, but also from X-ray radiation or gas emissions). For example, a film which covers a sensor device or also a protection glass can be provided as a protection device. Said protection device, in case it is arranged in the region of the treatment device, may also be thermally insulated.

The present invention is further directed at a method for treating the inner wall of containers, wherein the containers are transported by means of a transport device along a predetermined transport path and wherein the containers have a mouth opening, and wherein a treatment device is introduced through the mouth opening into the containers, and the treatment of an inner wall of the containers is monitored by means of a monitoring device.

According to the invention, the monitoring device monitors the treatment of the inner wall through the mouth opening. Said monitoring is particularly preferably carried out during a working operation of the apparatus, i.e., during a treatment process.

The monitoring device preferably monitors at least one emission of the treatment device and/or the physical consequences of said emission. The monitoring preferably takes place in such a way that at least one image of the interior of the container is recorded. In a preferred method, the monitoring takes place during a predetermined period. Preferably, the monitoring takes place during a period in which the treatment device is located inside the container and/or in which the container moves in its longitudinal direction relative to the treatment device.

The monitoring by the monitoring device preferably takes place continuously within a predetermined period.

Particularly preferably, the treatment process is a (plasma) coating process or a sterilization process of the inner wall of the containers. The treatment device preferably emits charge carriers, and in particular electrons, for carrying out the treatment process.

Further advantages and embodiments emerge from the accompanying drawing.

### DESCRIPTION OF THE DRAWINGS

In the drawing:

FIG. 1 shows a schematic representation of an apparatus according to the invention for treating containers.

### DETAILED DESCRIPTION OF THE INVENTION

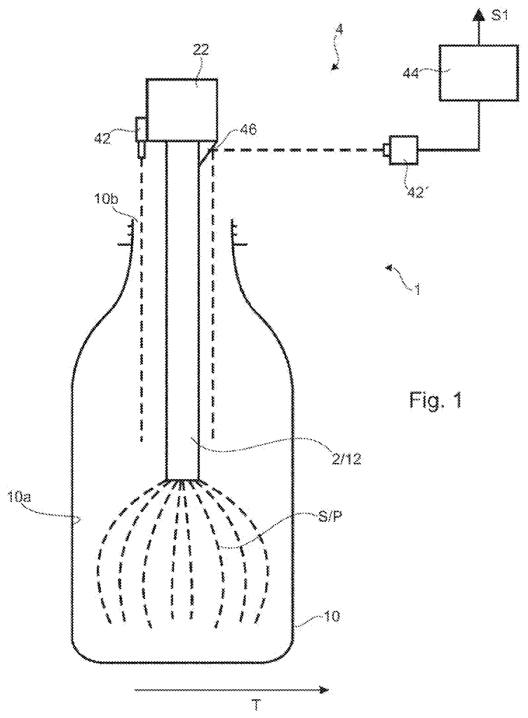

FIG. 1 schematically illustrates an apparatus 1 according to the invention. The reference sign 10 relates to a container whose inside wall 10a is treated—for example, sterilized or coated. For this purpose, a treatment device 2,12 is introduced through the mouth 10b into the interior of the container. The reference sign S/P identifies electron radiation or plasma which is applied to the inside wall 10a. Said radiation or the plasma S/P or effects caused by these can be detected with a suitable sensor device. In this way, the treatment process can (indirectly) be monitored too.

The reference sign 42 designates an image recording device which is arranged at an installation head 22 and can observe the presence of, for example, plasma through the mouth 10b of the container 10. Alternatively, a deflection element such as a mirror 46 may also be provided on the treatment head 22, so that the radiation can also be recorded by an image recording device 42′. Preferably, the image recording device can detect whether the treatment process is performed properly.

The reference sign 44 designates an evaluation device, such as an evaluation logic, which can output a signal S1. On the basis of said signal, it can be decided, for example, to discharge a specific container that was not adequately treated from a transport path. Said evaluation device can evaluate the image or images recorded by the image recording device (for example, compare with reference images) and determine whether the treatment process has taken place correctly. For example, the evaluation device can evaluate the image(s) captured by the image recording device with regard to the occurrence of radiation characteristic of the treatment process.

The image recording device 42, 42′, as well as the evaluation device 44, are components of a monitoring device, designated in its entirety by 4, for monitoring the treatment process.

The container shown in FIG. 1 is transported by a transport device (not shown) along a circular transport path, for example. During said transport, preferably the treatment of the container and the monitoring of the treatment is carried out.

The applicant reserves the right to claim all features disclosed in the application documents as essential to the invention, provided that they are novel over the prior art individually or in combination. It is also pointed out that features which can be advantageous in themselves are also described in the individual FIGURES. The person skilled in the art will immediately recognize that a particular feature described in a FIGURE can be advantageous even without the adoption of further features from this FIGURE. Furthermore, the person skilled in the art will recognize that advantages can also result from a combination of several features shown in individual or in different FIGURES.

1-11. (canceled)

12: An apparatus for treating the inner wall of containers, having a transport device configured to transport the containers along a predetermined transport path, wherein the containers have a mouth opening, wherein the apparatus has at least one treatment device which can be introduced into the containers through the mouth opening, wherein the apparatus has a monitoring device configured to monitor the treatment of the inner wall,
wherein
the monitoring device is configured to monitor the treatment of the inner wall through the mouth opening.

13: The apparatus according to claim 12,
wherein
the monitoring device has a sensor device which is configured for detecting electromagnetic radiation emerging from the interior of the container through the mouth opening.

14: The apparatus according to claim 13,
wherein
the sensor device is or has an image recording device which detects at least one image from the interior of the container through the mouth opening of the container.

15: The apparatus according to claim 13,
wherein
the sensor device configured to detect the radiation when the treatment device is introduced into the container.

16: The apparatus according to claim 12,
wherein
the treatment device has an emissions device which is configured to apply electron radiation or plasma to the inner wall.

17: The apparatus according to claim 12,
wherein
an image recording device and/or a radiation deflecting device is arranged on the treatment device.

18: The apparatus according to claim 12,
wherein
the monitoring device has a light guiding device.

19: The apparatus according to claim 12,
wherein
the monitoring device has a protection device configured to protect the sensor device from emissions occurring during the treatment.

20: The apparatus according to claim 12,
wherein
the apparatus has a cooling device configured to cool the treatment device.

21: A method for treating the inner wall of containers, wherein the containers are transported along a predetermined transport path of a transport device, and wherein the containers have a mouth opening, wherein a treatment device is introduced into the containers through the mouth opening, and the treatment of the inner wall is monitored by a monitoring device,
wherein
the monitoring device monitors the treatment of the inner wall through the mouth opening.

22: The method according to claim 21,
wherein
the monitoring is carried out in that at least one image of an interior of the container is recorded.
